(19) United States
(12) Plant Patent Application Publication
(10) Pub. No.: US 2024/0292773 P1
(43) Pub. Date: Aug. 29, 2024

(54) Campanula plant named `Midnight'

(50) ;

(71) Applicant: Hans A. Hansen, Zeeland, MI (US)

(72) Inventor: Hans A. Hansen, Zeeland, MI (US)

(73) Assignee: Walters Gardens Inc, Zeeland, MI (US)

(21) Appl. No.: 18/445,831

(22) Filed: Feb. 23, 2024

Related U.S. Application Data
(60) Provisional application No. 63/576,687, filed on Feb. 27, 2023.

(51) Int. Cl. A01H 6/26

(52) U.S. Cl. CPC A01H 6/264
USPC PLT/414

(57) ABSTRACT

The new and distinct Bellflower plant, Campanula plant named ‘Midnight’ with a dense, compact, mounded habit; cordate to deltoid medium-green foliage and numerous strong bluish-purple campanulate flowers for about 14 weeks beginning late spring and continuing until hard frost. The new plant is suitable for landscaping as a specimen, en masse, or in containers.

### CROSS-REFERENCE TO RELATED APPLICATIONS

This application claims the benefit of priority to U.S. Provisional Application No. 63/557,687, filed on Feb. 27, 2023, which is herein incorporated by reference in its entirety for all purposes.

Botanical designation: Campanula carpatica: Cultivar denomination: ‘Midnight’.

### STATEMENT REGARDING PRIOR DISCLOSURES UNDER 37 CFR 1.77(b)(6)

The new plant was first sold in Poland on Feb. 27, 2022, by Danziger who obtained the new plant and all information relating thereto, indirectly from the inventor. Information including a brief description and photographs of the claimed new plant was posted on a website operated by Walters Gardens, Inc. on Dec. 1, 2023. No plants of Campanula ‘Midnight’ have been sold in this country or anywhere in the world nor has any disclosure of the new plant been made more than one year prior to the filing date of the provisional application.

### BACKGROUND OF THE PLANT

The present invention relates to the new and distinct Bellflower herein also referred to as Campanula ‘Midnight’, by the cultivar name, ‘Midnight’, or as the new plant. The new plant was derived from a cross made on Jul. 8, 2016, from a plant in a greenhouse research facility in Zeeland, Michigan. The female parent was ‘Violet Teacups’ U.S. Plant Pat. No. 32,844 and the male parent was a proprietary, unreleased, and unnamed selection known only as 15-6-2 (not patented). The single selected seedling from this cross represented by ‘Midnight’ was selected from a group of seedlings by the inventor, isolated, compared in subsequent years to other Campanula and subsequently found to be different from all cultivars known to the discoverer, and eventually given the breeder code 16-1-1 prior to being assigned a cultivar epithet.

Asexual propagation at the same nursery in Zeeland, MI, USA by basal cuttings has shown ‘Midnight’ to be stable and reproduce true to type in successive generations since late June 2017.

### SUMMARY OF THE PLANT

Campanula ‘Midnight’ has not been observed in all possible environmental conditions. The phenotype may vary slightly with changes in environments such as light intensity, fertility, water availability, etc. without, however any variation in genotype.

Campanula ‘Midnight’ is distinct from all cultivars known to the inventor in the following traits:
1. Small, cordate to deltoid, serrate, medium-green foliage with compact height and dense mounded habit.
2. Heavily-branched with strong purple bell-shaped flowers clustered at the nodes and ends of stems.
3. Long flowering period for about 14 weeks beginning late spring and continuing until hard frost.

Plants of Campanula ‘Midnight’ are most similar to plants of the variety ‘Rapido Blue’ (not patented), ‘Blue Clips’ (not patented), and ‘Violet Teacups’. ‘Rapido Blue’ has a shorter flowering season, less flower coverage in peak flower, the flowers are larger and the flower center is light blue. ‘Blue Clips’ has a larger habit and slightly larger flowers with a shorter flowering season. ‘Violet Teacups’ has a larger habit and a more funnel-shaped flower with deeper cupping and a more violet and less bluish coloration.

### BRIEF DESCRIPTION OF THE DRAWINGS

The color drawings illustrate the overall characteristics of Campanula ‘Midnight’ as a five-year-old plant. The colors are as true as reasonably possible given the technology available. The color values may vary slightly depending on light intensity and quality.

FIG. 1 shows the new plant in a landscape environment.

FIG. 2 shows a close-up of the flowers and buds of the new plant.

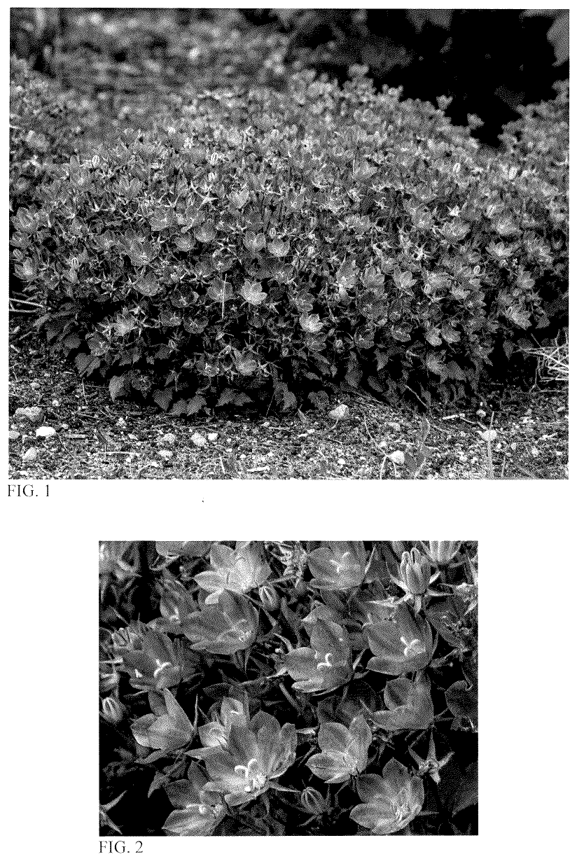

### DETAILED BOTANICAL DESCRIPTION

The following description is based on one and five-year-old plants growing in a full-sun trial garden and a partially shaded greenhouse in Zeeland, MI, USA. Except for ordinary dictionary color usage, color references are according to The Royal Horticultural Society Colour Chart, sixth edition, 2015. The new plant has not been observed in all possible growing conditions and may vary in phenotypic characteristics based on water availability, light conditions, fertilizer, temperatures, etc. without varying in genotypic characteristics.
Parentage: Female or seed parent is ‘Violet Teacups’; the male or pollen parent is 15-6-2;
Asexual propagation: Cuttings, about 12 to 20 days to initiate roots; time to finish in a one-gallon container about 12 weeks from an established 25 mm plug;
Plant habit: Rounded mound, herbaceous, perennial, winter-hardy; with heavily-branched, flower stems up to 48 cm wide and 28 cm tall; average about 42 cm across and about 20 cm tall; with about 24 well-branched stems per plant;
Roots: Finely branched; color nearest RHS NN155B;
Stems: Flexible; glabrous; thin; cylindrical with slight longitudinal ridges; to about 27 cm long and 2 mm diameter at base; with about 7 to 10 branches per stem; branches to about 13 cm long and 1 mm diameter; about 13 nodes per stem, internode spacing average about 2 cm;
Stem color: Uoung stems nearest RHS 137B, mature stems nearest RHS 146D toward base, no anthocyanins observed;
Node color: Proximally nearest RHS 146D, distally nearest RHS 137B;
Leaves: Cauline alternate; simple; ovate; acute apex; truncate to cordate base; margin serrate with 8 to 9 teeth per side, teeth about 0.5 mm to 2 mm long and 1 to 3 mm wide; adaxial and abaxial surfaces glabrous and matte; to about 33 mm long and 24 mm wide, average about 30 mm long and 21 mm wide;
Leaf color: Young expanding leaves adaxial nearest RHS 146B, abaxial nearest RHS 146B; mature leaves adaxial nearest RHS 137B, abaxial nearest RHS 147B;
Petiole: Thin; concavo-convex; glabrous both adaxial and abaxial; margin entire and sparsely hispidulous; to about 50 mm long and 2 mm wide at base;
Petiole color: Adaxial edge nearest RHS 137B and center nearest RHS 146B, abaxial between RHS 146D and RHS 145A;
Veins: Palmate; glabrous adaxial; puberulent, glabrate abaxial;
Vein color: Adaxial midrib between RHS 138C and RHS 146D and secondary veins nearest RHS 137A, abaxial midrib and secondary veins nearest RHS 138B, minor veins nearest RHS 137A;
Flower: Single; perfect; complete; campanulate; on terminal branches; to about 24 mm across, about 18 mm long; attitude outright to upright;
Inflorescence fragrance: Not detected;
Calyx: Divergent; to about 20 mm wide and extending about level with corolla base;
Sepals: Typically five; lanceolate; narrowly acute apex to apiculate; truncate base; margin micro-serrulate; glabrous adaxial and abaxial; about 10 mm long and about 2 mm wide at base;
Sepal color: Abaxial and adaxial nearest RHS 137B;
Flowering period: For about 14 weeks beginning'late spring and continuing until hard frost; producing about 120 flowers and 100 buds per plant at one time during peak flowering;
Flower longevity: About 5 to 7 days;
Flower buds: One day prior to opening—oblong; longitudinally channeled; rounded apex; about 12 mm long and 6 mm diameter;
Flower bud color: One day prior to opening—between RHS 85C and RHS N88D;
Peduncle: About 150 per plant; highly branched; cylindrical; glabrous; upright to outwardly attitude; flexible; about 2.5 mm diameter at base and 15 cm long;
Peduncle color: Nearest 138B;
Pedicel: cylindrical; Glabrous; thin, flexible; average about 8.5 cm long and 0.5 mm diameter;
Pedicel color: Between RHS 138A and RHS 138B;
Petals: Typically five, rarely six or seven; ovate; acute apex; basal 13 mm fused; glabrous adaxial and abaxial; about 18 mm long and 12 mm wide above fusion;
Petal color: Adaxial nearest RHS N88B; abaxial nearest RHS N88B;
Androecium: Typically five;
   Filament.—About 5 mm long and 1.5 mm wide at base and 0.1 mm thick, puberulent, flattened and adpressed along ovary in distal one-half; glabrous distally; color nearest RHS 157C;
   Anther.—Lanceolate; basifixed; about 4 mm long and 1.5 mm across; color nearest RHS 145C;
   Pollen.—Not abundant; color between RHS 163C and RHS 163D;
Gynoecium: Syncarpous; half-inferior; about 18 mm long;
   Style.—Cylindrical; puberulent distally, glabrous distally; about 10 mm long and 0.5 mm diameter; color nearest RHS 145C;
   Stigma.—Typically trifid, rarely quadra-fid or penta-fid; cochleate distally with maturity; about 6 mm long before curling, 3 mm long when curled, and 0.5 mm diameter; color nearest RHS 193C when young and nearest RHS 195B when mature;
Fruit: Oblong poricidal capsule; longitudinally carinate; 9 mm tall and 5 mm wide;
Fruit color: Nearest RHS N200A at maturity;
Seed: Flattened ellipsoidal; rounded apex and base; about 1 mm long and 0.5 mm across center;
Seed color: Variable, some seeds between RHS 200A and RHS N200A; Campanula ‘Midnight’ is tolerant of winter temperatures from USDA hardiness at least to zones 3 to 8. The new plant grows best in full-sun with good drainage and adequate moisture. It is not known to be tolerant of diseases and pest that are common to other Campanula cultivars.

I claim:

1. The new and distinct cultivar of Campanula plant named ‘Midnight’ as described and illustrated.
